(19) United States
(12) Patent Application Publication
(10) Pub. No.: US 2010/0311125 A1
(43) Pub. Date: Dec. 9, 2010

(54) Method for Liquefying and Saccharifying Starch Quickly

(75) Inventors: Xingyao Xiong, Changsha (CN); Keqin Wang, Changsha (CN); Xiaojun Su, Changsha (CN)

Correspondence Address: DAVID AND RAYMOND PATENT FIRM, 108 N. YNEZ AVE., SUITE 128, MONTEREY PARK, CA, 91754, US

(73) Assignee: HUNAN AGRICUL TURAL UNIVERSITY, Changsha (CN)

(21) Appl. No.: 12/811,378

(22) PCT Filed: Sep. 19, 2008
(86) PCT No.: PCT/CN08/72419

§ 371 Date: Jul. 1, 2010

(51) Int. Cl. C12P 19/00

(52) U.S. Cl. 435/72

(57) ABSTRACT

A method for liquefying and saccharifying starch quickly includes the steps of: irradiating starch with 0.25×10⁶˜1.5×10⁶ Gy ⁶⁰Co-γ rays or electron accelerator, putting the irradiated starch into a fermenting tank and adding cold water, stirring mechanically to forma solution, adding a little amount of starch saccharifying enzyme to saccharifying the starch under acid condition.

### BACKGROUND OF THE PRESENT INVENTION

1. Field of Invention

The present invention relates to, and more particularly to a liquefaction and saccharification method for a starch object under a low temperature condition while requires a shortened period of time.

2. Description of Related Arts

Conventional saccharification of a starch object makes use of a tank to boil or heat the starch object under high temperature. The starch object is mixed with water at a ratio of 1:37 in a stirring device for stirring at 55˜60° C., then is transferred to the tank by using a reciprocating pump in which steam is used as a heating media to heat up the mixture within the tank at a heating temperature around 140˜150° C. The mixture is collected from a top end of the tank and is guided to a ripening device for allowing ripening for 90 minutes to obtain a resulting ripen starch object which is in the form of sugar paste such as mash extract. The ripen starch object is then transported to a vacuum container for cooling. After cooling, the ripen starch object is transferred to a saccharization tank in which glucoamylase is added for saccharification for 50 minutes at 60° C. The ripen starch object is then cooled to 30° C. by spray drying, which is then ready for processing for fermentation. The convention method requires or employs a lot of standards or parameters throughout the process. For example, the saccharification parameters or standards are as follows: a heating temperature at 145° C., a ripening period of 90 minutes and a concentration of 19° Bx, and a reducing sugar at 2%; the parameters of a saccharification temperature at 60° C., a saccharification period of 50 minutes, glucoamylase of 100U, saccharification concentration of 16˜18° Bx, pH 2.0˜2.5, and a reducing sugar at 4˜6%. The major drawback of the conventional method of saccharification under high temperature is that the cost is high, the ethanol production is low due to over decomposition of the starch object, the difficulty in cleaning the saccharization tank is high, the effectiveness of spray cooling is low, the waste of cooled water, and etc.

### SUMMARY OF THE PRESENT INVENTION

The invention is advantageous in that it provides a liquefaction and saccharification method for a starch object under a low temperature condition and a short period of time in which the starch object is treated by irradiation using cobalt-60 gamma rays (⁶⁰Co-γ rays) or by direct radiation processing using an electron accelerating irradiation chamber. Compared to conventional method, the step involving boiling or steaming under high temperature condition is eliminated, the time of processing is shortened, and the energy consumption is lowered while the enzyme for starch liquefaction such as amylase is no longer required. Treating the starch object under radiation of high energy level can effectively break down the glucosidic bonds in the starch object such that a high percentage of the starch object is then capable of being subjected to saccharification directly, while the quantity requirement of enzyme for saccharification such as glucoamylase is reduced by two-third in quantity. Therefore, the process is highly effective and efficient to facilitate industrial production and processing.

Additional advantages and features of the invention will become apparent from the description which follows, and may be realized by means of the instrumentalities and combinations particular point out in the appended claims.

According to the present invention, the foregoing and other objects and advantages are attained by a method of liquefying and saccharifying a starch object, comprising the following steps. The starch object is treated by irradiation using ⁶⁰Co-γ ray (cobalt-60 gamma ray) or electron accelerating irradiation chamber to obtained a treated starch object, wherein a radiation dose of irradiation is 0.25×10⁶˜1.5×10⁶ Gy. After the radiation treatment, the treated starch object is placed in a fermentation processor. A predetermined amount of water is added into the fermentation processor, where a ratio of the treated starch object and the water is 1:10 by weight. The treated starch object is dissolved with the water through mechanical stirring in the fermentation processor to form a dissolved starch object. An acidity (pH value) of the dissolved starch object is then adjusted to 4˜4.5 to form a pretreated starch object. An enzyme for saccharification such as glucoamylase in the amount of 20˜30 IU is added to the pretreated starch object for saccharification, where a treatment time for saccharification is 1 hour and a treatment temperature for saccharification is 60° C., to obtain a saccharized product containing about 10% reducing sugar which is suitable for direct processing of fermentation. In treating the starch object by irradiation using ⁶⁰Co-γ ray (cobalt-60 gamma ray) or electron accelerating irradiation chamber, the present invention provides great advantageous effect over the conventional method which requires boiling or steaming a starch object under controllably high temperature condition and uses enzyme for liquefaction such as amylase which are temperature sensitive and destroyable under temperature. In particular, the method of the present invention not only involves only low heating level and simple equipment requirements while simplifying the number of steps as well as the complexity of steps, but also is direct and effective in application for industrial production.

These and other objectives, features, and advantages of the present invention will become apparent from the following detailed description, the accompanying drawings, and the appended claims.

### DETAILED DESCRIPTION OF THE PREFERRED EMBODIMENT

The present invention provides a method of liquefying and saccharifying a starch object, comprising the steps of: treating the starch object by irradiation using ⁶⁰Co-γ ray (cobalt-60 gamma ray) or electron accelerating irradiation chamber to obtained a treated starch object, wherein a radiation dose of irradiation is 0.25×10⁶˜1.5×10⁶ Gy; then, placing the treated starch object in a fermentation processor; adding a predetermined amount of water into the fermentation processor, where a ratio of the treated starch object and the water is 1:10 by weight; dissolving the treated starch object with the water, preferably cold water, through mechanical stirring in the fermentation processor to form a dissolved starch object; adjusting an acidity (pH value) of the dissolved starch object to 4˜4.5 to form a pretreated starch object; adding an enzyme for saccharification such as glucoamylase in the amount of 20˜30 IU to the pretreated starch object for saccharification, where a treatment time for saccharification is 1 hour and a treatment temperature for saccharification is 60° C., to obtain a saccharized product containing about 10% reducing sugar which is suitable for direct processing of fermentation. In treating the starch object by irradiation using ⁶⁰Co-γ ray (cobalt-60 gamma ray) or electron accelerating irradiation chamber, the present invention provides great advantageous effect over the conventional method which requires boiling or steaming a starch object under controllably high temperature condition and uses enzyme for liquefaction such as amylase which are temperature sensitive and destroyable under temperature. In particular, the method of the present invention not only involves only low heating level and simple equipment requirements while simplifying the number of steps as well as the complexity of steps, but also is direct and effective in application for industrial production.

According to a preferred embodiment of the method of liquefying and saccharifying a starch object of the present invention, the starch object which is 150 kg in a package is used. The starch object is treated by irradiation using ⁶⁰Co-γ ray with a source radioactivity of 20 millicurie and a radiation dose of 3×10⁵ Gy to obtain a treated starch object. Then the treated starch object is placed in a fermentation processor such as a fermentation tank into which a predetermined amount of water is added, where a ratio of the treated starch object and the water is 1:10 by weight, and is dissolved in the water through mechanical stirring in the fermentation processor to form a dissolved starch object. Then, an acidity (pH value) of the dissolved starch object is adjusted to 4˜4.5 to form a pretreated starch object, which is then arranged for saccharification by adding an enzyme for saccharification such as glucoamylase in the amount of 20˜30 IU, where a treatment time for saccharification is 1 hour and a treatment temperature for saccharification is 60° C., to obtain a saccharized product. The saccharized product obtained from the above process which is in the form of sugar paste contains approximately 10% reducing sugar which can be used directly for fermentation. In other words, it is unnecessary to use different equipments such as saccharization tank and fermentation tank for liquefaction, saccharification and fermentation and hence eliminating any possible waste of materials during transfer between different containers and equipment. In addition, the treated starch object is dry, easy to handle and manipulate which increases the ease of handling and accuracy, making the present invention highly effective and efficient for use in industrial production.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of liquefying and saccharifying a starch object, comprising the steps of:
treating the starch object by irradiation using cobalt-60 gamma ray (⁶⁰Co-γ ray) or electron accelerating irradiation chamber to obtained a treated starch object, wherein a radiation dose of irradiation is 0.25×10⁶˜1.5×10⁶ Gy;
placing the treated starch object in a fermentation processor;
adding a predetermined amount of water into the fermentation processor, wherein a ratio of the treated starch object and the water is 1:10 by weight;
dissolving the treated starch object with the water through stirring in the fermentation processor to form a dissolved starch object;
adjusting a pH value of the dissolved starch object to 4˜4.5 to form a pretreated starch object; and
adding glucoamylase in the amount of 20˜30 IU to the pretreated starch object for saccharification, wherein a treatment time for saccharification is 1 hour and a treatment temperature for saccharification is 60° C.
